(19) United States
(12) Patent Application Publication
(10) Pub. No.: US 2025/0318736 A1
(43) Pub. Date: Oct. 16, 2025

(54) MEDICAL DEVICES

(71) Applicant: Heba BEVAN, Bromley (GB)

(72) Inventor: Heba BEVAN, Bromley (GB)

(21) Appl. No.: 19/240,151

(22) Filed: Jun. 17, 2025

Related U.S. Application Data
(63) Continuation-in-part of application No. 16/622,759, filed on Dec. 13, 2019, now abandoned.

(30) Foreign Application Priority Data
Jun. 14, 2017 (GB) 1709435.0

(51) Int. Cl. A61B 5/0205; A61B 5/00; A61B 5/01; A61B 5/024; A61B 5/1455; G16H 50/20

(52) U.S. Cl. CPC A61B 5/02055; A61B 5/01; A61B 5/02427; A61B 5/14552; A61B 5/443; A61B 5/6801; A61B 5/7271; G16H 50/20; A61B 2560/0214; A61B 2560/0252; A61B 2560/0462; A61B 2562/0266

(57) ABSTRACT

An infection sensing system that comprises a wearable sensor device configured to be fastened onto skin. The sensor device has a sensors including a temperature sensor, a heart rate sensor, and a reflective SpO2 sensor. A multivariate time-series analysis neural network trained to perform temporal pattern recognition, generates an output that predicts infection. The neural network processes time series data that extends over a duration of at least one hour. The time series data representing body temperature, heart rate, and SpO2 level input into the multivariate time-series analysis neural network comprises data that comprises substantially simultaneous measurements for at least the heart rate and SpO2 level.

### FIELD OF THE INVENTION

This application is a Continuation-in-part of U.S. application Ser. No. 16/622,759 filed Dec. 13, 2019, which is a national stage of International Application No. PCT/GB2018/051647 filed Jun. 14, 2018, which claims priority to Great Britain Application No. 1709435.0 filed Jun. 14, 2017, each of which is hereby incorporated herein by reference in its entirety.

### BACKGROUND TO THE INVENTION

Typically when at home and an infection is suspected a measurement is made of body temperature. If this is elevated then potentially an infection is present and a visit to a doctor or hospital may be made. However, it would be useful to be able to improve upon such approaches, in particular for children and old people.

### SUMMARY OF THE INVENTION

In an example embodiment there is described an infection sensing system, the system comprising: a sensor device configured to be fastened onto the skin, the sensor device comprising a plurality of sensors; and a processor coupled to the sensors. The sensors can include at least: a temperature sensor to read skin temperature as a proxy for body temperature; and a heart rate sensor, and may also include an SpO2 sensor. The processor is configured to: input data from the sensors and determine data representing body temperature and heart rate; and identify a combination of: (i) a greater than threshold temperature fluctuation in said body temperature, and (ii) a greater than threshold heart rate, and wherein (i) and (ii) are present for greater than a threshold time duration. The processor can also process SpO2 measurements from the SpO2 sensor. In responsive to the identification the processor can store and/or output data indicating infection.

In broad terms, it has been recognised that when a person (or animal) develops an infection there is generally an effect on the heart rate as well as the temperature. More particularly the heart rate is elevated, and there is apparently an interaction between body temperature and heart rate which results in a temperature fluctuation rather than a simple temperature rise. In addition, frequently the skin moisture level increases as the infected person sweats. Thus a significantly increased confidence of detecting an infection is achieved by detecting one, two or all of these factors in combination. Furthermore, it is desirable to establish that these conditions are present for an extended period of time, for example, at least one hour, two hours of three hours. Optionally, however, an increased skin moisture level may be detected in combination with a temperature fluctuation instead of an elevated heart rate. SpO2 can also decrease.

Some particular example systems for processing data in this way are described later. In some implementations it is advantageous to collect and process time series data from the sensors using a trained multivariate time-series analysis neural network, to generate an output that indicates predicted infection.

However in practice some difficulties have been found with this approach, in particular in obtaining consistent and reliable infection prediction. The inventor has identified and found a solution to this problem. In more detail, it might be thought that when collecting data over long periods, or order an hour or a few hours, the precise timing of the data collection would not matter. Counterintuitively, however, it appears that it is important to capture the data processed by the multivariate time-series analysis neural network almost simultaneously, at least as regards heart rate and SpO2 data, despite the vast difference in timescales. In this context “almost simultaneously” means on a timescale of milliseconds. Without wishing to be bound by theory it appears that there can be differential effects in the measured data resulting from user or patient motion, in particular that associated with breathing. The motions discussed here are different to gross motions of a user that, say, an accelerometer can be used to detect and control. The motions are relatively subtle but appear to have an effect on the captured data such that, when this data is processed by a trained multivariate time-series analysis neural network, they in effect add noise that can affect the correct operation of the neural network and, in particular, the reliability with which it predicts infection.

In one aspect there is therefore described an infection sensing system that comprises a wearable sensor device configured to be fastened onto skin. The sensor device has a plurality of sensors, and a processor coupled to the sensors. The sensors comprise at least a temperature sensor to read skin temperature as a proxy for body temperature, a heart rate sensor, and an SpO2 sensor, in particular a reflective SpO2 sensor arranged to perform a one-sided measurement of oxygen saturation.

The processor is configured to input time series data from said sensors and determine data representing body temperature, heart rate, and SpO2 level, and to process the data representing body temperature, heart rate, and SpO2 level using a multivariate time- series analysis neural network trained to perform temporal pattern recognition, to generate an output indicating infection.

The multivariate time-series analysis neural network is configured to process time series data that extends over a duration of at least the threshold time duration. The threshold duration can be at least one hour, e.g. greater than 2, 4, 6, or 12 hours. In implementations the time series data representing body temperature, heart rate, and SpO2 level input into the multivariate time-series analysis neural network comprises data that comprises simultaneous measurements for at least the heart rate and SpO2 level. Here the definition of “simultaneous” is that the simultaneous measurements are simultaneous to better than 100 milliseconds or 10 milliseconds.

In implementations making such simultaneous measurements can help to ensure reliability of the infection prediction by the trained multivariate time-series analysis neural network. This is counterintuitive as the time series extends over an hour or more, some four to six orders or magnitude longer than the measurement timescale; and is further counterintuitive as the time scales of heart rate measurement are typically longer than 100 ms.

In some implementations each of the temperature fluctuation, raised heart rate and optionally raised skin moisture level are required to be present over the same threshold time duration, but in principle different time durations could be employed for the different sensed conditions. It is also desirable to make repeated measurements over a period of time, for example, at least two or three such measurements at intervals of at least of two, three, four, five, six hours or more. By combining these indications a substantially increased confidence that there is potentially an infection can be established. For example in infants an elevated temperature may merely relate to teething.

The determination of whether a particular parameter is greater than a threshold level may be made in a number of different ways. For example, a parameter may be integrated over time, or an average value of the parameter over time may be determined, or a single instance of the parameter being greater than the threshold may be detected, or there may be a requirement for a plurality of measurements of the parameter being greater than the threshold. In a similar way whether or not there is greater than a threshold temperature fluctuation may be determined using any of these approaches. Such a threshold temperature fluctuation may require the temperature to be greater than a maximum value and/or less than a minimum value and/or may require a change in temperature greater than a threshold temperature range, and/or greater than an integrated temperature variation over time. The threshold time durations for which the heart rate/moisture level/temperature fluctuation are required to be present may be the same or different for the different parameters. In embodiments the data indicating infection may indicate a potential degree of infection, for example dependent upon one or more of the determined heart rate/moisture level/temperature fluctuation.

In principle different types of detection may be distinguished responsive to a determination of which parameters are greater than their respective threshold levels. For example, a greater than threshold temperature fluctuation, potentially irrespective of heart rate, may indicate an infection of a first type whilst a combination of a greater than threshold heart rate and a greater than threshold skin moisture level, without necessarily a greater than threshold temperature fluctuation, may indicate an infection of a second type. In related embodiments, the processor control code is configured to identify first and second combinations of parameters with different respective parameter threshold levels in order to distinguish between first and second types or categories of infection. The stored/output infection data may then distinguish between these different types or categories of infection.

In some preferred embodiments the device power supply is driven by a difference between the body temperature and the environmental temperature, for example employing a thermoelectric generator such as a Seebeck effect device. For example the sensor device may comprise an enclosure with a sensing surface comprising a reduced thickness face or membrane to touch the skin and an external face opposite the sensing surface. A circuit substrate mounting the sensing system electronic circuitry may be located on a rear face of the enclosure, with thermal insulation between this and the sensing surface; the thermoelectric power generating device may span the sensing and external faces.

Alternatively, the power supply may be charged by the user and/or environment in some other manner, for example by the user's motion (although preferably the user is required to be stationary during the measurement process). Typically the electrical power produced by such a thermoelectric generator is very small (the power source may provide less than 1 μA current). In embodiments, therefore, the device may remain in a sleep mode, only waking at intervals to make one or more measurements; and/or the device may incorporate a receiver, in particular an RF receiver, which is used to wake the device up to transmit the recorded data. Alternatively, to save power, the device may rely upon a wired rather than a wireless interface to extract the infection data.

In some embodiments the device may be provided with a strap to mount the device on the body; thus the device may be in the form of a watch. However, in some preferred embodiments the device has the form of a plaster, in that the device may be stuck onto a region of the body where sensing is optimal—for example in the vicinity of a carotid artery. In such embodiments the mounting portion may comprise an adhesive to allow the device to be attached to the skin. Preferably the device is then fabricated on a flexible substrate such as a flexible circuit board. Then preferably (where possible) flexible components are employed, for example a flexible thermoelectric generator and so forth. Typically the processor will not be flexible, but is relatively small. Preferred embodiments of such devices are disposable and may be single-use.

In embodiments the skin moisture sensor may be implemented in a variety of ways, for example measuring electrical resistance/impedance using electrodes in contact with the skin and/or by measuring capacitance. The heart rate sensor may be an optical sensor but in some preferred embodiments the heart rate sensor comprises one or both of a pressure sensor and an accelerometer. In preferred embodiments signals from both a 30 pressure sensor and an accelerometer are combined, for example by determining a heart rate from each and then averaging. Preferred embodiments of the device include one or more accelerometers in order to identify when the body is in a rest state. Preferably at least heart rate is measured preferentially or only when the body is determined to be substantially in such a rest state (i.e. not when active/moving).

The infection sensing system may also make use of chemical and/or gas sensors to identify the presence of an infection. Accordingly, in some embodiments the sensors further comprise one or more of a chemical sensor or a gas sensor, and the processor is configured to identify that a combination of (i) a greater than threshold temperature fluctuation in said body temperature, (ii) a greater than threshold heart rate, optionally (iii) a greater than threshold skin moisture level, and (iv) a greater than threshold concentration of chemical indicative of a medical condition, are present for a duration greater than said threshold time duration.

The chemical indicative of a medical condition may be a by-product of a medical condition, for instance, an expression of a gene associated with cancer, or glucose or one or more ketones associated with diabetes. The chemical sensor may be configured to measure one or more chemicals on the skin. The gas sensor may be configured to measure one or more chemicals in the air around the system (adjacent to the skin).

In embodiments a supervised or unsupervised machine learning algorithm may be employed to process one or more of body temperature data, heart rate data, and skin moisture data (all as described further below). A supervised machine learning algorithm may operate in combination with data indicating an actual infection (for example from a physician), to allow the machine learning algorithm to provide a prediction of infection. The supervised machine learning algorithm may be used to train the neural network. An unsupervised machine learning algorithm may operate on the input data to learn patterns in the data indicative of infection without the need for feedback from a physician. Optionally the machine learning may be performed in hardware and/or software in the sensor device, for example on a CPU of the sensor device; alternatively the algorithm may be partially or wholly implemented elsewhere, for example in a base station or on a remote server. Accordingly, measurements from the infection sensing system may be output to an external processing system to analyse the measurements to determine whether an infection is present in accordance with the infection identification steps described herein.

In a related aspect the invention provides a method of sensing infection using an infection sensing system, in particular as claimed in any preceding claim, the method comprising: measuring body temperature and/or heart rate and skin moisture level; and identifying a combination of: (i) a greater than threshold temperature fluctuation and/or a combination of: (ii) a greater than threshold heart rate, and (iii) optionally a greater than threshold skin moisture level; wherein (i), (ii), and optionally (iii), are present for greater than a threshold time duration; and responsive to said identification storing and/or outputting data indicating infection.

Embodiments of the system may include a processor coupled to the sensors and to memory storing processor control code. The processor control code may comprise code to control the processor to process the input data as describe. However in some preferred embodiments the processor includes some dedicated hardware to process the data, and thus some or all of the processing may be implemented in such hardware.

Thus embodiments of both the system may include a processor in combination with working memory and non-volatile memory including memory storing processor control code to implement the above described functions. The processor(s) employed in the device may operate under the control of stored program code, or may comprise dedicated hardware implemented in electronic circuitry, or may comprise a combination of some dedicated hardware modules and some systems under program control. Additionally or alternatively, however, the data processing described above may be implemented partially or wholly in dedicated hardware such as a programmable gate array and/or ASIC (application specific integrated circuit). As the skilled person will be aware, the functionality of such a device may be distributed between multiple hardware elements in wired or wireless communication with one another; some or all processing may be performed in hardware, some or all processing may be performed in software.

The invention further provides processor control code to implement the above-described devices and methods, for example on a general purpose computer system or on a mobile device, or on a digital signal processor (DSP). The code is provided on a non-transitory physical data carrier such as a disk, CD- or DVD-ROM, programmed memory such as non-volatile memory (eg Flash) or read-only memory (Firmware). Code (and/or data) to implement embodiments of the invention may comprise source, object or executable code in a conventional programming language (interpreted or compiled) such as C, or assembly code, or code for a hardware description language. As the skilled person will appreciate such code and/or data may be distributed between a plurality of coupled components in communication with one another.

### Urine-Flow Based Diagnostic Systems

In a further aspect the invention provides a urine-flow based diagnostic system, the system comprising: a sensor device for use in a toilet bowl or urinal so as to intersect a stream of urine, the sensor device comprising a urine stream flow sensor; and a processor coupled to said urine stream flow sensor; and wherein the processor is configured to: determine, from said urine stream flow sensor, a flow rate parameter dependent on a sensed urine flow; and responsive to said flow rate parameter, store and/or output data indicating presence of a potential medical condition.

Embodiments of such a device are particularly useful in identifying potential urinary tract infections (UTIs) although they are not limited to detecting infections of this type. For example the device can also be used to detect a prostate condition such as prostate cancer. In practice there are other conditions which can mimic the presence of infection or prostate cancer (for example Benign Prostatic Hyperplasia may mimic prostate cancer). The device does not diagnose a condition, but rather provides an indication of a potential problem which should then be followed up with a proper medical examination.

Detecting a potential UTI in the elderly is difficult because their immune system may not mount an effective response and thus they may not exhibit fever; instead the symptoms may be similar to dementia. Nonetheless the elderly are vulnerable to UTIs which, if left untreated, can cause serious complications such as kidney damage/failure and sepsis. It is also difficult to identify UTIs in children.

The system may comprise just the sensor device, which may include the processor, and/or the sensor device may be wired or wirelessly coupled to the or another processor in a separate device, for example a mobile device or base station. Additionally or alternatively the system may include a remote server, for example in the cloud. The base station and/or remote server may perform additional processing on processed data derived from the sensor device, for improved accuracy.

In embodiments a supervised or unsupervised machine learning algorithm may be employed to process one or more of urine flow rate data, urine flow/pressure data, flow peak count data, flow peak timing data, and urine colour data (all as described further below). A supervised machine learning algorithm may operate in combination with data from a physician on an actually diagnosed condition (to supervise the learning), to allow the machine learning algorithm to provide a prediction of one or more medical conditions. The supervised machine learning algorithm may be used to train a neural network. An unsupervised machine learning algorithm may operate on the input data to learn patterns in or classify the data which patterns or classification(s) are indicative of one or more medical conditions without the need for feedback from a physician. Optionally the machine learning may be performed in hardware and/or software in the sensor device, for example on a CPU of the sensor device.

The flow rate parameter may comprise raw or processed data from one or more pressure sensors; if multiple pressure sensors are employed an average of the sensors or of a selected one or more of the sensors may be used. In embodiments the urine flow rate is determined from sensed pressure by a applying calibration parameter; this may depend upon the sensor/sensor plate geometry and may be determined by experiment for a particular sensor or sensor combination. Examples of sensor configurations are described later.

Embodiments of the device we describe detect a potential UTI or other condition from measuring flow of the urine stream. An infection or other medical condition may be detected by a reduction in urine flow rate, for example, to less than 10, 8 or 5 ml/s—a figure which is surprisingly relatively independent of age. More particularly, however, intermittent flow rate appears to be characteristic of infection, and the duration between periods of flow appears to relate to the degree of infection, longer periods being associated with a greater level of infection.

Embodiments of the device are configured to detect, from the flow rate parameter, when the urine flow is intermittent. For example, in embodiments this may be achieved by detecting a greater than threshold fluctuation in flow rate, or of a sensed parameter dependent upon the flow rate. Additionally or alternatively, periods of relatively higher and lower flow rate may be distinguished, for example by comparing the flow rate with a threshold (which may optionally exhibit hysteresis), flow rates (or rate-sensing parameters) having a value less than this threshold denoting reduced or ceased flow and flow rates higher than this value denoting adequate flow. For example in such instances the threshold is chosen to be below the reduced flow rate expected when infection is present, so as to distinguish between reduced flow rate and close to stopped flow. Detection of such intermittent flow may be used to store and/or output data indicating an infection or other condition. In embodiments the degree of infection may be determined, for example by classifying the duration between periods of intermittent flow into one of a plurality of categories determining one of a (corresponding) categories of level of infection.

Preferred embodiments of the system process the flow rate parameter (for example pressure sensor data) to distinguish peaks in the parameter. Typically this involves some filtering of the data and a implementation of peak detection procedure, an example of which is described later.

The system may be configured to identify a final part of the flow from a flow pattern or flow rate of the flow, and to disregard this. In embodiments peaks are detected down to a threshold maximum peak height, for example 0.2 ml/sec flow rate. In embodiments the final one or two peaks are then discarded.

In embodiments one or more of the following items of information may then be derived: a count of the number of peaks; a maximum urine flow rate (or pressure) over a peak (or some value representing this); an interval between one or more peaks (denoted T1 later); and a peak duration (denoted T3 later). A peak duration may be determined from a duration between corresponding flow rates, for example minimum flow rates, to either side of the peak.

Preferably the system also determines a time duration of a period of reduced or substantially zero flow between one or more peaks (denoted T2 later). This may be determined from a duration between corresponding flow rates to either side of a minimum between adjacent peaks. The corresponding flow rates may be a threshold level above the minimum flow rate; that is the T2 duration may be defined as the time between flow rates defined by the minimum flow rate plus a delta. In this way the time duration between peaks may be meaningfully defined whether or not the flow rate reduced to zero between peaks.

The skilled person will appreciate that where references are to flow rate, a value dependent upon or proportional to flow rate, for example a sensed pressure (or value dependent thereupon) may likewise be employed. Where it is desired to employ a peak-dependent timing value as described above, where multiple peaks are present an average value may be employed.

The system may be configured to identify when a peak flow rate, more particularly the flow rate of the initial or highest detected peak, is less than a threshold value, for example 20 ml/sec, 15 ml/sec or, preferably, 10 ml/sec, to identify the presence of a potential medical condition. Optionally two threshold flow rates may be employed, a first, higher threshold to detect the potential presence of a prostate condition such as prostate cancer, and a second, lower threshold to detect the potential presence of an infection. The higher threshold may be, for example 20 ml/sec, 15 ml/sec or, preferably, 10 ml/sec; the lower threshold may be 15 ml/sec, 10 ml/sec or, preferably, 5 ml/sec. Optionally the flow rate may be integrated to determine a total volume flow.

Depending upon the use case, multiple total flow measurements may be combined (where, say, the measurements relate to the same individual), for example to determine a morning or afternoon or daily total volume flow.

Some preferred embodiments of the system include an optical sensor to detect a “colour” of the urine, that is to distinguish between clear and dark urine. Such a sensor may comprise, for example, a light source (eg LED) and detector. In broad terms this allows embodiments of the system to detect the presence of blood in the urine. However, as previously noted, embodiments of the system do not provide a diagnosis but merely indicates a need for further investigation—for example eating beetroot can give a false positive. In preferred embodiments the colour detection is combined with other detected signals of an infection as described above and below; in embodiments multiple such signals are required for the system to indicate the presence of a potential infection.

In preferred embodiments the system determines the number of peaks and uses this to identify the presence of a potential medical condition. More particularly, the presence of multiple peaks indicates a potential medical condition, and a count of the number of peaks can distinguish between a prostate condition such as prostate cancer and an infection—one, two or a small number of peaks is indicative of a prostate condition such as prostate cancer, particularly in combination with other indicators, such as flow rate, as described herein; whilst two or more than two peaks is indicative of an infection. (This is particularly where the final one or two small peaks are disregarded). In broad terms, where an infection is present there are more, smaller peaks than for potential prostate cancer. Where an infection and a prostate condition such as prostate cancer are both present the number of peaks tends to correspond to that for a prostate condition rather to that for infection.

Experimental work has determined that when an infection is present the duration of a peak, T3, may be below a threshold value; and/or T2 may be greater than a threshold value (for example greater than 0.5 secs or greater than 1 second); and/or T1 (as measured, say, between the first two peaks, or as an average over peaks) may be lower than a predetermined threshold value.

Experimental work has determined that when a prostate condition such as prostate cancer is present T3, may be greater than a threshold value; and/or T2 may be less than a threshold value (for example less than 0.5 secs or substantially zero); and/or T1 (as measured, say, between the first two peaks, or as an average over peaks) may be greater than a predetermined threshold value.

It will be appreciated that the differences in the responses of one or more of these timings may be employed to differentiate between an infection and a prostate condition. Optionally a ratio of T2 to T3, such as T2/T3, may also be used to identify changes in T2 and/or T3 and/or to determine the presence of a condition and/or to differentiate between infection and a prostate condition (T2/T3 is higher for a prostate condition than for an infection).

Optionally the timing and/or flow rate data and/or peak count data may be used to determine the potential degree of a medical condition such as an infection

The system may analyse one or more chemicals in the urine or in gas from the urine. In further embodiments: the sensor device further comprises a chemical sensor, and said processor is further configured to identify presence of the potential medical condition responsive to a detected concentration of a chemical indicative of a medical condition within said urine; and/or said sensor device further comprises a gas sensor and said processor is further configured to identify presence of the potential medical condition responsive to a detected concentration of a chemical indicative of a medical condition in gas deriving from said urine.

Certain chemicals and smells are indicative of a medical condition. The chemical sensor may be configured to detect one or more gene products (e.g. RNA or protein) of genes associated with cancer. For instance, prostate-specific antigen (PSA) is a protein produced exclusively by prostate cells. PSA is a glycoprotein enzyme. There is a blood test to measure PSA level in men. This may help to detect early prostate cancer. Having said this, the blood test is not accurate.

An increased level of PSA increases the chance that the subject has prostate cancer. 13% of men over 55 have a PSA level of greater than 4 ng/ml. Having said this, an increased level of PSA does not necessarily mean that the subject has prostate cancer. An elevated level can also be due to other conditions, such as benign enlargement of the prostate (BPH), a urinary tract infection or a prostate infection.

Accordingly, by detecting an increased level of PSA within urine (or within gas deriving from the urine), embodiments may be able to identify the presence of a medical condition for further investigation by a trained medical professional.

Other chemicals that may be detected include expressions of TMPRSS2:ERG or PCA3, both of which are indicative of prostate cancer, and/or an increased level of ketones or glucose within urine, which can be indicative of diabetes.

The systems described herein may indicate the detection of a medical condition when a concentration of the chemical is detected that exceeds a predefined threshold.

The chemical or gas sensors may be configured to identify chemicals that are not indicative of a medical condition, but that may cause a false positive in the system. For instance, the presence of beetroot products (e.g. betalain pigments) within urine can cause discolouration that the system may mischaracterise as blood. Accordingly, the system may be configured to detect one or more pigments within the urine that are not associated with a medical condition in order to avoid mischaracterisation. Upon identification (e.g. upon measuring a concentration that exceeds a predefined threshold), the system may inhibit any response that derives from a measurement of the colour of the urine, or may compensation for any colour measurement to account for the measured pigment. The chemical and gas sensors may also identify an increased concentration of the urine, which may be indicative of dehydration. This may be further indicated by an increased concentration of sulphur within the urine or within gas emanating from the urine.

Optionally the system may include a local alert device such as a display device or alarm, which may be located in a bathroom (restroom), for example adjacent a toilet, to alert a user to the presence of a potential medical condition.

In embodiments the flow sensor comprises a plurality of spatially distributed flow sensors, in separate housings and/or in multiple housings (sensor enclosures). One device may be in wired or wireless communication with one or more other devices, to enable sensed data to be combined, for example averaged.

In one embodiment a sensor device may comprise an enclosure with a sensing surface comprising a moveable plate or membrane mounted on a set of one or more pressure sensors to provide distributed flow sensors. Signals from multiple sensors may be combined by averaging or the like. A pressure sensor may comprise an accelerometer in combination with a spring, to detect movement of the membrane or plate. Embodiments of the device determine flow rate or a proxy for flow rate such as pressure or an accelerometer signal, as time series data.

In one embodiment a sensor device may comprise a ring-shaped enclosure mounting a pressure-sensing surface comprising a plate or membrane having a set of apertures through which urine may pass. Such a device may be mounted across the neck of a toilet bowl (and may additionally be used to sense the passing of a kidney stone). Such a device may be used in combination with one or more other sensor devices placed at one or more other locations in a toilet bowl, for example on a forward or rearward surface of the bowl. Optionally the ring-shaped enclosure may be suspended from one or more other sensor devices which may themselves be fastened, for example glued onto the toilet bowl. The sensor device with the ring-shaped enclosure may be attached to one or more other sensor devices, for example by means of a releasable, for example magnetically fastened, attachment such as an attachment line or connection. Signals from these multiple sensing devices may be combined.

It has been found that conventional flushing systems in toilets and urinals do not always completely clean away contaminants and waste from the sensors after use. This can lead to inaccurate measurements when the system is next used. For instance, the system may incorrectly detect blood within the urine if some blood is left over from the previous use.

In one embodiment, the system further comprises a cleaning system comprising a reservoir configured to hold fluid and a release mechanism for releasing at least some of the fluid to clean at least part of the sensor device. This allows the system to clean contaminants and waste away from the sensor device after use. Cleaning the sensor device may comprise flushing away, dissolving and/or chemically breaking down (e.g. chemically cleaning away) contaminants. The fluid may by cleaning fluid. The fluid may comprise any fluid suitable for washing away contaminants or waste, such as water, bleach, or solvent(s). The reservoir may be built into the system which, in turn, may be retrofitted to a toilet or urinal. Accordingly the flushing mechanism may be in addition to a flushing mechanism of the toilet or urinal.

According to an embodiment the processor is configured to control the release mechanism to release the fluid after urine has been detected. The system may detect when a person has finished urinating and release the cleaning fluid to clean the system in order to prepare the system for future use. The system may determine that a person has finished urinating when no urine flow has been detected for a predetermined period of time or when an end of use input has been triggered. This end of use input may be the toilet or urinal being flushed (e.g. the detection of water) or through an input from the user such as via a button, pull-chord, or via an input from a device connected to the system (e.g. a mobile phone).

According to a further embodiment the system comprises a fluid collection mechanism configured to collect the fluid over one or more sensing apparatus of the system after the cleaning fluid has been released from the reservoir. This may be in the form of an actuating barrier that may be raised so that fluid may be collected over the one or more sensing apparatus, and that may subsequently be lowered in order to release the fluid. The fluid collection mechanism may release the fluid after a predetermined period of time, or after the one or more sensors indicate that they are sufficiently clean (e.g. that they are within a predefined range of normal operating parameters). This provides the fluid additional time to clean the one or more sensors. The actuating barrier may be the movable plate mentioned above. The one or more sensors may comprise a colour sensor, a chemical sensor and/or a gas sensor.

The time at which the urination takes place may also indicate a medical condition. For instance, an increase in the frequency of urination (the frequency that the subject needs to urinate) and/or an increase in the frequency of urination during a period of the day or night may indicate a medical condition. For instance, increased frequency of urination at night may be indicative of a urinary tract infection, bladder infection or diabetes (among other conditions). Accordingly, in one embodiment the processor is configured to record a time at which the urine flow is detected and, responsive to one or more of an increased frequency of urination or an increased frequency of urination during a predetermined period of the day-night cycle, store and/or output data indicating the presence of a medical condition.

Multiple different users may make use of the system. Accordingly, it may be advantageous to reset the system so that measurements are not shared between different users. According to a further embodiment the system comprises an input means and the processor is configured to identify the presence of a new user in response to an input and, in response to the identification of the new user, establish a new set of measurements for the new user. The input may be the detection of a flush by the user (e.g. via the flow sensor) or via an input from the user such as from a button, pull-chord, or via a device connected to the system (e.g. a mobile phone).

Where multiple users use the system, it can be advantageous to track each user's measurements across multiple uses. This can allow the system to build up a profile for the user so that anomalous results can be detected over time. According to an embodiment the processor is configured to identify the new user in response to receipt of a unique user identifier and to associate any subsequent measurements and identifications of medical conditions with the unique user identifier until the presence of another user is identified. The unique user identifier may be received via an input from a radio frequency identification (RFID) reader configured to read an RFID tag or magnetic reader configured to read a magnetic tag.

The infection sensing systems and the urine-flow based diagnostic systems described herein may be configured to communicate with each other, e.g. via wireless communication. This can allow the systems to share measurements to allow more accurate identification of medical conditions. According to an embodiment there is provided an infection sensing system further as described herein further comprising a wireless communication module, wherein the processor is configured to trigger one or more measurements by one or more of the sensors in response to an input from a urine-flow based diagnostic system as described herein indicating that the user of the infection sensing system is urinating. This allows the systems to work together to take additional measurements during urination. These additional measurements may include one or more of heart rate, skin temperature, skin moisture, chemical measurements and gas measurements.

Where multiple sensors are present the system (code) may be configured to distinguish between genders flow dependent upon a sensed spatial distribution of the urine flow. For example where sensors are located at the corners of a sensing plate a male-pattern flow tends to activate just one or two sensors whereas a female pattern flow tends to be more distributed, activating more or all the sensors. Where a male-pattern flow is detected signals from non-activated sensors (sensors with outputs less than a threshold level) may be disregarded. Distinguishing genders can be helpful, for example in distinguishing or ruling out a prostate-related condition.

Optionally urine flow may be sensed using a multiple-axis, for example 3-axis accelerometer, accelerometer data from these multiple axes being combined to determine a parameter reflecting the urine flow rate (helping to make the device flow-direction-insensitive). For example the accelerometer output may be integrated over each of the three (X, Y and Z) directions.

Optionally, multiple axis accelerometer data may be employed to distinguish the gender of the source of the urine flow, by determining one or more characteristics of the accelerometer data and using this to distinguish between genders. For example, the standard deviation of the measured acceleration along one or preferably multiple axes, for example X, Y and/or Z axes, may be employed to distinguish between male and female flows. Optionally the flow rate may be employed to determine an age bracket of the flow originator; alternatively if a device is to be used by a particular age demographic, such as the elderly, this information may be pre-programmed into the device in order to set thresholds and the like.

The system may comprise at least two sensors, a first sensor to detect initiation of the flow and a second sensor to detect flow rate. Operation of the second sensor may be initiated by the first sensor detecting initiation of the flow. Thus the first sensor may comprise a temperature sensor or, in some preferred embodiments, a pressure sensor, and this may be employed to wake up the device/second sensor when flow begins. Alternatively, the first sensor may comprise, for example, an optical sensor such as a colourimetric sensor. Additionally or alternatively, the second sensor may comprise a microphone or similar acoustic vibration sensor. In this case, it is advantageous only to turn on, or begin collecting data from, the acoustic sensor once flow has been detected by, for example, a detected change in pressure and/or change in temperature. This helps to avoid the sensor detecting background noise. It appears that an acoustic sensor is able to detect flow with increased accuracy. It will be appreciated that it is not essential to control operation of an acoustic sensor with another sensor.

As previously mentioned, in embodiments the device is configured to distinguish and disregard the final part of the urine flow, for example based upon the flow pattern and/or flow rate. This helps to make the infection detection more robust. One way in which the final part of the flow can be detected is by the typically reduced pressure of this portion of the flow. Another technique is by the flow pattern, which is somewhat gender-specific—in females the flow apparently ceases whereas in males there is a stop-flow-stop pattern which can be distinguished from the previously mentioned intermittent flow.

As previously mentioned, optionally, embodiments of the device may sense parameters of the urine such as temperature and/or colour using a temperature and/or optical sensor. A temperature sensor can be employed to determine whether the urine temperature (which reflects the body temperature) is too high or too low (elevated or depressed). An optical sensor can be employed to determine whether or not there is blood in the urine or, more generally, whether the urine is overly dark. Data from one or more such further sensors may be combined with data from the flow rate sensing, for example to indicate infection when any of these conditions is detected, or to indicate infection only when more than one of these conditions is present. Although, generally, the sensing device detects urinary tract infections, other infections such as kidney infections or kidney stones, may also be detected.

In a related aspect the invention provides a method of sensing presence of a potential medical condition using a urine sensing device including a urine stream flow sensor, the method comprising: determining, from said flow sensor, a flow rate parameter dependent on sensed urine flow rate; and responsive to said flow rate parameter, storing and/or outputting data indicating presence of a potential medical condition.

Embodiments of the system may include a processor coupled to the sensor device and to memory storing processor control code. The processor control code may comprise code to control the processor to process the input data as described. However in some preferred embodiments the processor includes some dedicated hardware to process the data, and thus some or all of the processing may be implemented in such hardware.

Thus embodiments of the system may include a processor in combination with working memory and non-volatile memory including memory storing processor control code to implement the above described functions. The processor(s) employed in the device may operate under the control of stored program code, or may comprise dedicated hardware implemented in electronic circuitry, or may comprise a combination of some dedicated hardware modules and some systems under program control. Additionally or alternatively, however, the data processing described above may be implemented partially or wholly in dedicated hardware such as a programmable gate array and/or ASIC (application specific integrated circuit). As the skilled person will be aware, the functionality of such a device may be distributed between multiple hardware elements in wired or wireless communication with one another; some or all processing may be performed in hardware, some or all processing may be performed in software.

The invention further provides processor control code to implement the above-described devices and methods, for example on a general purpose computer system or on a mobile device, or on a digital signal processor (DSP). The code is provided on a non-transitory physical data carrier such as a disk, CD-or DVD-ROM, programmed memory such as non-volatile memory (eg Flash) or read-only memory (Firmware). Code (and/or data) to implement embodiments of the invention may comprise source, object or executable code in a conventional programming language (interpreted or compiled) such as C, or assembly code, or code for a hardware description language. As the skilled person will appreciate such code and/or data may be distributed between a plurality of coupled components in communication with one another.

### BRIEF DESCRIPTION OF THE DRAWINGS

These and other aspects of the invention will now be further described, by way of example only, with reference to the accompanying figures, in which:

FIGS. 1a to 1c show, respectively, a block diagram of a infection sensing device according to a first embodiment of the invention, a vertical cross section through a physical embodiment of the device, and a schematic vertical cross section through a infection sensing device according to a second embodiment of the invention;

FIG. 2a shows a flow diagram of a procedure for controlling a device of FIG. 1;

FIG. 2b shows a block diagram of another example infection sensing system;

FIG. 3 shows a block diagram of a urine infection sensing device according to an embodiment of the invention;

FIG. 4 shows a flow diagram of a procedure for controlling the device of FIG. 3;

FIGS. 5a and 5b show preferred embodiments of a urine flow sensor device and its mounting;

FIG. 6 shows a block diagram of a urine-flow based diagnostic system according to an embodiment of the invention; and

FIG. 7 shows a flow diagram illustrating operation of a urine-flow based diagnostic system according to an embodiment of the invention.

### DETAILED DESCRIPTION OF PREFERRED EMBODIMENT

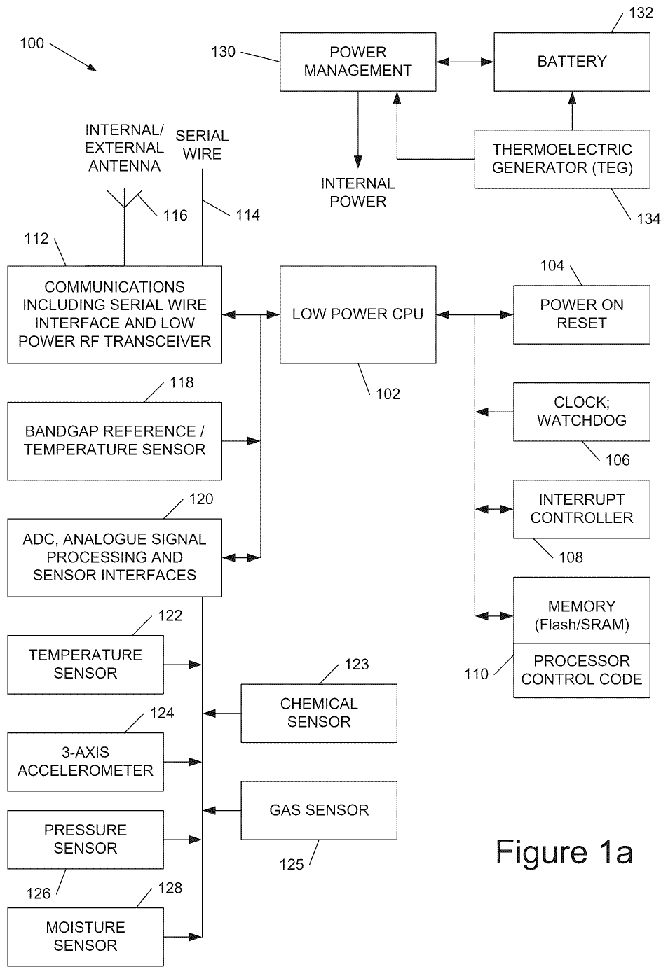

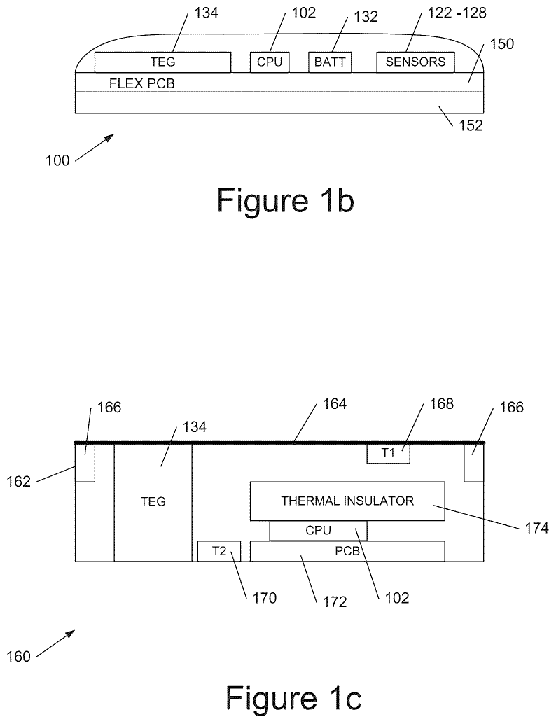

Referring to FIG. 1a, this shows an embodiment of a wearable infection sensing device 100. The device comprises a low power CPU 102 coupled to power on reset circuitry 104, to a clock (and to watchdog clock circuitry) 106, to an (optional) interrupt controller 108, to working memory (RAM), and to non-volatile memory (Flash) 110 storing processor control code for controlling CPU 102. The CPU is also coupled to a communications interface 112 including a serial wire interface 114 and/or a low power RF transceiver coupled to an internal and/or external antenna 116. CPU 102 is also coupled to a temperature sensor 108, for example employing a bandgap reference, and to an analogue-to-digital convertor and signal processing interface 120 for interfacing to a set of sensors. These sensors comprise, in embodiments, a further temperature sensor 122, a chemical sensor 123, a 3-axis accelerometer 124, a gas sensor 125, a pressure sensor 126, and a moisture sensor 128. Power management circuitry 130 is coupled to a battery (or supercapacitor) 132, and to a thermoelectric power generator (TEG) 134 or other energy harvester. In operation thermoelectric generator 134 charges battery 132 which provides internal power to the device based on a small difference between the body temperature and environment temperature.

The gas sensor 125 may be a chemical gas sensor and/or an electrochemical nose (e-nose). An example of a gas sensor is the Figaro TGS2602-B00 Air Contaminants Gas Sensor. The gas sensor 125 may be configured to detect the concentration of one or more chemicals in gas in or around the toilet bowl or urinal. The chemical sensor 123 may be configured to detect the concentration of one or more chemicals within the urine. This may be through one or more receptors configured to interact with one or more chemicals to detect the one or more chemicals and measure the associated concentration.

FIG. 1b shows a cross section through a physical embodiment of the device, in the illustrated example fabricated on flexible circuit board 150 bearing an adhesive layer 152 so that the device can be attached to the skin, preferably adjacent to an artery. The device includes a CPU, battery, sensors and thermoelectric generator as previously described, preferably covered by a conformal coating 154. Preferably as many of the components as practical are flexible including, for example, the thermoelectric generator and battery. The gas sensor 125 may be at least partially exposed to the surrounding air. The chemical sensor 123 may be exposed to the skin of the subject.

FIG. 1c shows a cross section through a further example device 160. In this device an enclosure 162 has a front plate or membrane 164 (typical thickness ˜1 mm), which is pressure sensitive and can thus be used to detect a heartbeat. To detect pressure the plate is mounted on an accelerometer-spring combination at each corner 166. A first temperature sensor T1 168 is mounted on (thermally coupled to) this plate to measure skin/body temperature and a second temperature sensor T2 179 is mounted on the opposite (external) face of the device, to measure an environmental temperature. This enables compensation for the environmental temperature. A PCB 172 mounts a CPU 102 and other electronics and, importantly, is thermally insulated from sensor T1 by insulation 174. In embodiments a thermoelectric generator 134 spans the skin-contacting and external faces of the device. Optionally a skin moisture sensor (such as one or more electrodes, not shown) may be provided on plate 164. Optionally, a chemical sensor (not shown) may be provided on plate 164. Optionally, a gas sensor (not shown) may be provided on the opposite (external) face of the device (to the plate 164) to measure the concentration of one or chemicals in the surrounding air.

Elements of the example embodiment of FIGS. 1b may be included in the example embodiment of FIGS. 1c, and vice-versa. For example a reduced thickness face/membrane as described with reference to FIG. 1b may be included in the example of FIG. 1c, and so forth.

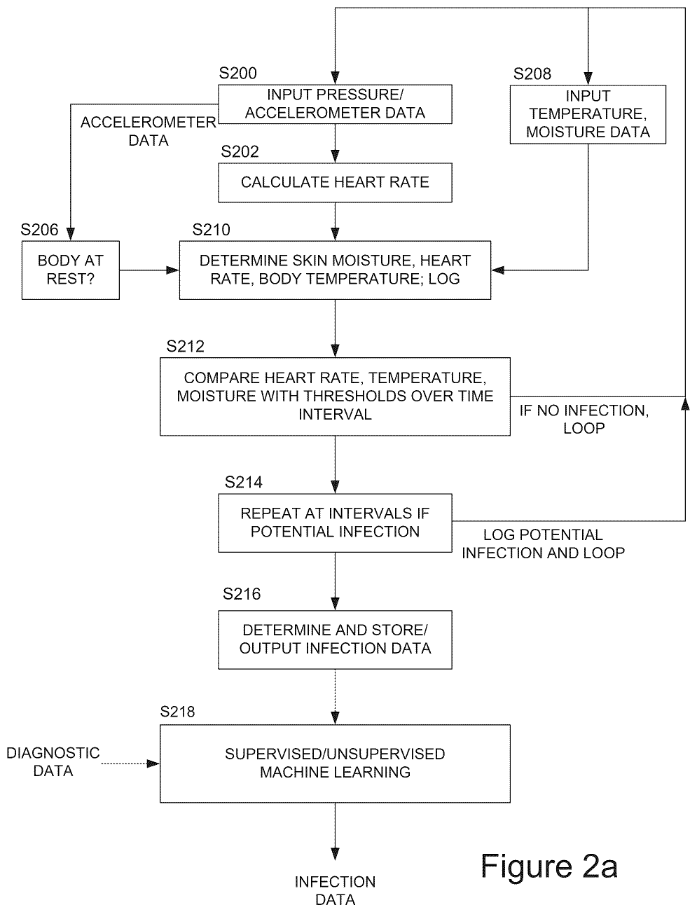

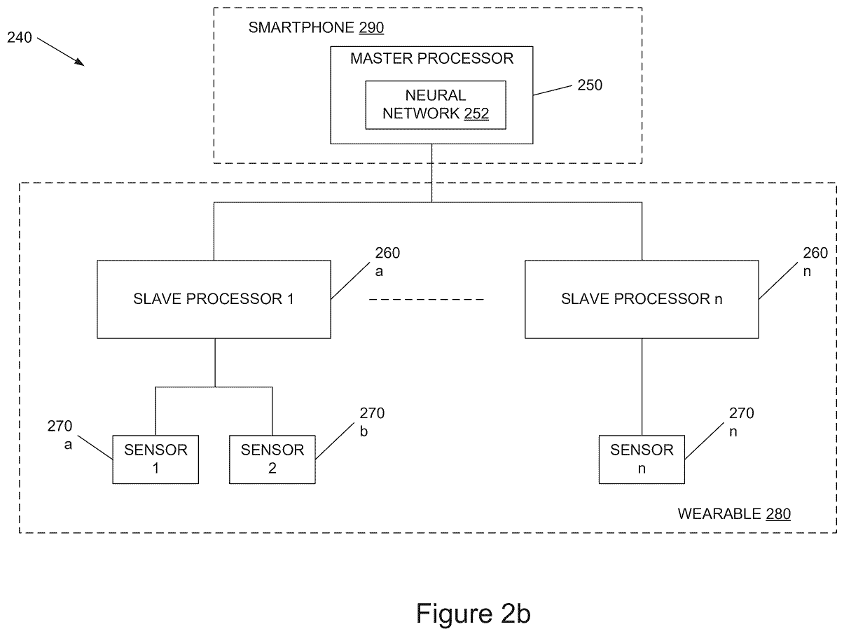

Referring next to FIG. 2a, this shows a flow diagram of an embodiment of a procedure for the CPU of the wearable device of FIG. 1. Thus at step S200 the procedure inputs pressure and accelerometer data and combines this data to calculate a heart rate for the wearer (S202). The procedure also uses the accelerometer data to determine whether or not the person is at rest (S206), disregarding data collected when the person is moving and, preferably, for some duration after the person has stopped moving.

The procedure also collects data from the temperature sensors and humidity sensor (S208) and processes the temperature sensor data to determine an estimated body temperature, and processes the humidity sensor data to determine an estimated skin moisture level (S210); at this point the procedure also has the heart rate data. In embodiments, the raw and/or processed sensor data may optionally be logged for later interrogation. The procedure then applies criteria to the heart rate, temperature and moisture levels to determine whether or not there is a potential infection (S212).

In further embodiments, chemical concentration data (such as from a chemical or gas sensor) may also be input at step S208 and compared to a respective threshold in step S212 to determine whether there is a potential infection.

If no potential infection is identified the procedure loops back to re-check the sensor data; if there is a potential infection the procedure logs the potential infection and loops back (S214) to repeat the test at intervals, for example, every few hours.

At step S212 embodiments of the procedure identify the combination of a temperature fluctuation between high and low temperature values in combination with a raised heart rate, where the elevated heart rate may be determined with respect to an absolute threshold in beats per minute (bpm) or with respect to a heart rate dependent upon an individual or category of individual, for example an age-dependent heart rate. Optionally multiple combinations of heart rate and temperature fluctuations may be identified. For example, a first set of conditions may comprise detecting, during a 3 hour period, that the heart rate goes above 71 bpm on at least one occasion and that there is at least one high-low temperature fluctuation (whilst the body is in its rest state). Such a condition may determine infection with a first probability, for example 75%. A second set of conditions may, for example, define that during a period of 1 hour the heart rate at some point exceeds 75 bpm and there is a high-low temperature fluctuation, again whilst the body is in its rest state; this may define a higher probability of infection, for example 88%. A requirement for an increased skin moisture level may also be included. A third example condition may detect an increased heart rate and an increased moisture level whilst the body temperature is approximately normal; a fourth type of condition may identify a temperature fluctuation without requiring an increased heart rate or increased skin moisture level. One or more of these types of condition may be employed; preferably all are determined when the body is in its rest state.

Once infection data has been determined this data is stored and at some convenient time output on a wired or wireless connection (S216). The infection data may comprise a single bit defining whether or not an infection has been identified, and/or it may include information identifying a level and/or probability of infection and/or it may include data identifying a type of infection or set of conditions identified (as described above), and/or the data may include raw or processed sensor data, for example so that a medical practitioner may review the historically measured heart rate and/or temperature and/or moisture data.

Optionally (un)supervised machine learning may be applied to the raw and/or processed data and/or output data (S218) to provide a more robust indication of potential infection. The machine learning may be implemented on the device of FIG. 1a, 1b or 1c, or partially or wholly remotely, for example on a base station or remote server. When training the neural network using a supervised training technique training data may be collected by collecting time series data of the type processed by the neural network, from a cohort of patients, where the infection status of the patients is later determined by conventional means to act as ground truth data for training the system.

Whilst the above embodiments discuss infection sensing, alternative embodiments may be configured to detect a medical condition. For instance, an increase in glucose measured by a chemical sensor may indicate diabetes. Equally, the processor may be configured to detect other medical conditions, such as cancer, through the detection of associated chemicals (e.g. one or more expressions of genes associated with cancer).

The infection sensing system may be configured to reset measurements after use. This allows the system to be used with a different user. The reset ensures that measurements from a previous user are not erroneously associated with a new user. The reset may be in response to the system being removed from the skin of a user (identified by changes in the detected skin moisture, temperature and pressure). Alternatively, the reset may be via using a magnetic wave from a magnetic tag, and RFID signal from an RFID tag, from a wireless signal (e.g. a Bluetooth signal or a command from a wireless base station), or from a reset input such as a button.

Various different alternative sensing arrangements and combinations of conditions are contemplated within the scope of the invention. For example, in one aspect the invention may include just a temperature sensor and may detect a greater than threshold variation in temperature over a period of time. Alternatively the invention may include sense heart rate and moisture sensors but omit a temperature sensor, and may detect a combination of increased heart rate and increased moisture over a period of time. As previously stated, the moisture sensor is optional but preferable.

As previously described, the device itself may provide a signal or may communicate by means of a wired or wireless connection with a base unit or base station, for example a mobile phone, desktop computer or the like. The skilled person will appreciate that, in the various different aspects of the invention, the infection data may be provided via a network such as a mobile phone network or the internet, for example to alert a carer. Additionally or alternatively, an alarm may be triggered should an infection level greater than the threshold and/or a persistent infection be detected.

FIG. 2b shows a block diagram of another example infection sensing system 240. Elements of the previously described examples, e.g. as described with reference to FIG. 1b and or 1c, may be included in the example infection sensing system 240 of FIGS. 2b, and vice-versa.

The example infection sensing system 240 comprises a wearable sensor device 280 configured to be attached or fastened onto skin, e.g. using a strap. The wearable sensor device 280 has a plurality of sensors 270a . . . n. These can be as described elsewhere herein; advantageously there are as described below. The system also comprises a processor coupled to the sensors 270. In the illustrated example the processor comprises a master processor 250 and one or more slave processors 260a . . . n.

In the illustrated example the sensors 270a,b,n comprise at least a temperature sensor to read skin temperature as a proxy for body temperature, a heart rate sensor, and an SpO2 sensor. Advantageously the SpO2 is a reflective SpO2 sensor arranged to perform a one-sided measurement of oxygen saturation (i.e. both the light source and the sensor are on the same side of the measuring site).

The processor 250, 260, is configured, e.g. by appropriate software stored in non-volatile memory, to input time series data from the sensors, to determine data representing body temperature, heart rate, and SpO2 level.

The processor 250, 260, is also configured to process the data representing body temperature, heart rate, and SpO2 level using a multivariate time-series analysis neural network 252, to generate an output indicating infection. The multivariate time-series analysis neural network has trained to perform temporal pattern recognition e.g. using a supervised learning technique based on ground truth training data comprising examples of uses with and without an infection.

The multivariate time-series analysis neural network 252 is configured to process time series data that extends over a duration of at least one hour, e.g. more than 2, 3, 4, 6, or 12 hours. In implementations the time series data representing body temperature, heart rate, and SpO2 level input into the multivariate time-series analysis neural network comprises data that comprises “simultaneous” measurements for at least the heart rate and SpO2 level. The definition of “simultaneous” is that the simultaneous measurements are simultaneous to better than 100 milliseconds or 10 milliseconds. This can help to ensure reliability of the infection prediction, counterintuitively, given the vast difference in timescales, and even though the time scales of the time series heart rate measurement are typically longer than 100 ms.

In the context of measuring heart rate (either instantaneous or average), the time of making a measurement for defining whether or not measurements of heart rate and SpO2 are simultaneous can be defined in various ways. As one example, the measurement time can be defined by reference to a particular point in the heart beat cycle, e.g. a particular point on an ECG. Then whatever point is chosen, this point can be maintained so that it is consistent for all the measurements so that the measurements are consistently simultaneous to whatever particular definition of simultaneous is chosen. As another example the multivariate time-series analysis neural network can have been trained using training data comprising the measurements that the neural network is configured to process, e.g. temperature, heart rate, SpO2, and so forth, and using ground truth infection data. In this case the measurements can be consistently simultaneous using the same definition of simultaneous that was adopted during training, more specifically by defining a particular point in the heart beat cycle to use as a time reference point at which heart rate (either instantaneous or average) is measured. This same time reference point can then be used when determining a time of other measurements such as SpO2, gas level, and optionally moisture, and temperature.

In some implementations a master-slave processor system of the type illustrated in FIG. 2b can be used to facilitate obtaining simultaneous measurements, by decoupling obtaining the measurements from processing the measurements using the neural network

As previously described, the master-slave processor system comprises the master processor 250 coupled to the one or more slave processors 260. This can be a wired or wireless coupling. For example where the master processor is located in a smartphone, laptop, or other similar device, 290, and the slave processor(s) 260 are located in a wearable device which as a watch-style device, the master and slave processors can have a wireless coupling.

The one or more slave processors 260 are coupled to the sensors 270, and the master processor 250 is configured to implement the multivariate time-series analysis neural network 252. The master processor can be configured to control the one or more slave processors to perform the simultaneous measurements.

In some implementations the master processor is implemented on a smartphone, laptop computer or other similar device 290. The one or more slave processors are implemented on a wearable device 280, e.g. a watch-style device, comprising the plurality of sensors and the enclosure, e.g. as previously described. As well as facilitating the simultaneous measurements, this also facilitates the use of lower power (reduced computational or memory capacity and/or reduced power consumption) slave processors and a relatively higher power master processor, contributing, e.g. to increased battery life for the wearable.

In some implementations the multivariate time-series analysis neural network 252 comprises a temporal convolutional network or recurrent neural network, e.g. LSTM. Such an approach, as compared with some other possibilities, can facilitate processing potentially noisy time series data.

In some implementations of the system the sensors of the wearable device 280 include a gas sensor configured to measure a level of NO or NO2 emitted by a wearer of the wearable device. The input time series data can then include the level of NO or the level of NO2, and the multivariate time-series analysis neural network can also process the level of NO or the level of NO2 to generate the output predicting infection.

Rather than use a MEMS sensor the system can use a compound semiconductor gas sensor. Such a compound semiconductor gas sensor can be based on a metal oxide (MOX) or on a III-V or II-VI semiconductor. An advantage if such a sensor in combination with processing by the multivariate time-series analysis neural network is that such sensors can have relatively low noise when detecting NO or NO2, as well as being very sensitive, e.g. approaching ppb level detection.

In some implementations a compound semiconductor gas sensor can also or instead be used to measure a level of CO2 emitted by a wearer of the wearable device. The input time series data can then include the level of CO2 and the multivariate time-series analysis neural network can be further configured to process the level of CO2; to generate the infection predicting output. CO2 sensing can be difficult, especially where there are size and power consumption constraints. Use of a compound semiconductor gas sensor can help overcome these constraints. As another example, a compound semiconductor gas sensor for CO2 detection can use an array of carbon nanotubes.

### Urine-Based Sensing

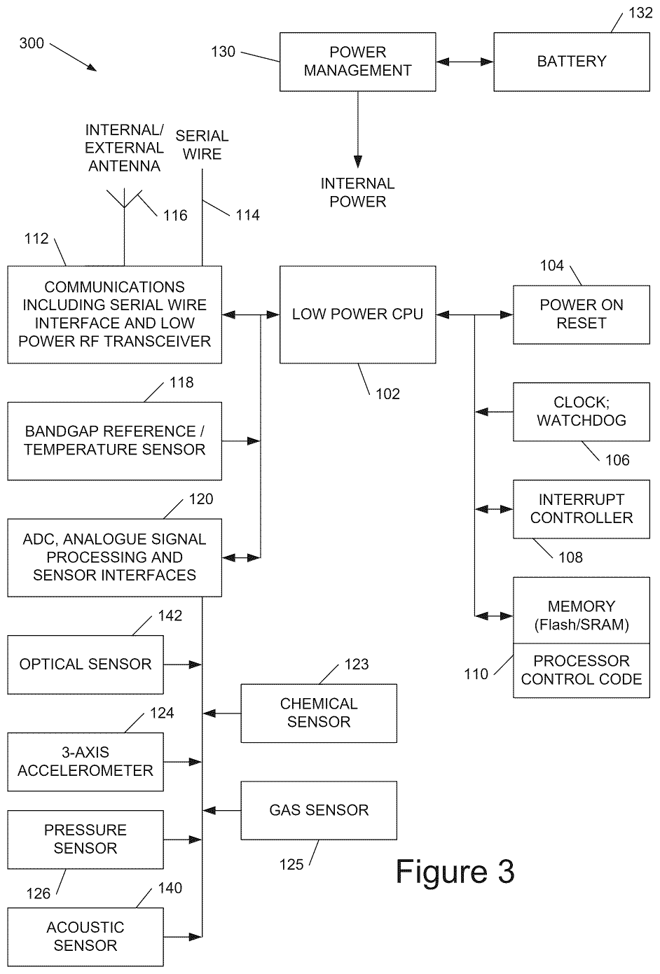

FIG. 3 shows a block diagram of a urine sensing device 300, in which like elements to those of FIG. 1 are indicated by like reference numerals. As can be seen, a different set of sensors may be employed including, for example, an optional acoustic sensor or microphone 140 and an optional optical sensor 142 for example for colourimetric measurements. The stored processor control code will also be different to that of FIG. 1. The device of FIG. 3 may be powered by a rechargeable or replaceable battery or may be wireless-chargeable.

FIG. 3b illustrates an example physical unit illustrating that the circuitry may be contained in a small plastic case 350, sealed so that it is waterproof, and bearing a hook 352 or some other mounting means to facilitate mounting unit within a toilet bowl or urinal.

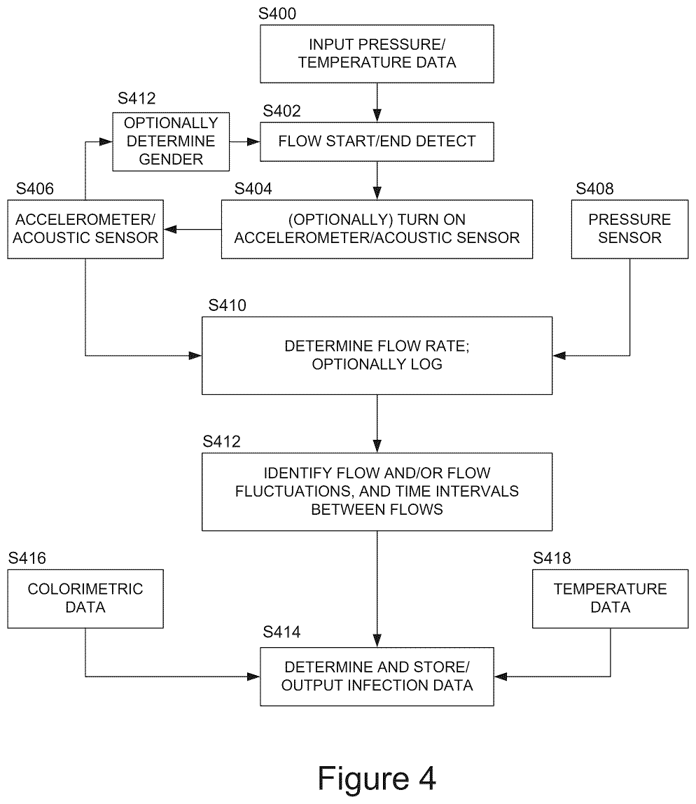

FIG. 4 shows a flow diagram of an example procedure implemented by the device at FIG. 3. Thus at step 400 the procedure inputs pressure and/or temperature data and uses this to detect the start of a urine flow onto the device (S402). The time at which the urine flow starts may be detected and recorded. This can be used to identify a medical condition, for instance, based on frequency of urination or frequency of urination during a predefined period of time (time of day/night).

Once flow has been detected the procedure begins gathering data from the accelerometer and/or turns on the acoustic sensor (microphone), S404. Data is then collected from the accelerometer and/or acoustic sensor (S406) and, in embodiments, also from the pressure sensor (S408). These data are used to determine the urine flow rate (S410), for example by integrating the X, Y and Z-axis accelerometer signals to determine a direction-insensitive flow rate and then optionally combining this, for example by weighted average, with a flow rate from the pressure sensor, to determine time series flow rate data.

Optionally the accelerometer data may also be provided to a routine S412 which estimates the gender, for example from the standard deviation of the X-,Y- and Z-accelerometer signal components (greater for males than for females). This information may provide information to the flow-end detection procedure S402 as the male and female end of flow behaviour differs. In broad terms, therefore, embodiments of the device may estimate the user's gender, and then adapt the operation of the device in response to the user's gender, in particular to increase the accuracy of the data obtained. Although in embodiments this information is applied to end of flow detection a skilled person will appreciate that knowledge of gender may be used in other ways to improve the accuracy of the measurements.

Once flow rate has been determined, the procedure identifies the absolute flow rate as a low flow rate, for example less than 10 ml/s, can indicate infection (S412). Additionally or alternatively the procedure identifies flow fluctuations. In some preferred embodiments the procedure identifies when the flow falls to substantially zero, and preferably also determines a duration or an average duration between such intervals and/or counts a number of such intervals. In particular, the duration between points at which the flow approaches zero is useful for identifying a potential infection. More particularly, where an infection is present there may be an extended period of time for which the flow is close to zero (below a threshold level). Thus there may be short intervals of flow punctuated by relatively long delays. In embodiments, therefore, the device may time the duration of one or more of such delays between intervals of flow and determine a delay or an average delay. In broad terms, the longer the delay between intervals of flow the greater the likelihood of infection and/or greater the degree of infection. The skilled person will appreciate that there are many ways to define a threshold level of flow in order to count or time durations of low flow or intervals between duration periods of flow.

In further embodiments, a gas and/or chemical sensor is/are turned on at step S404 and measure chemical concentration at step S406. This concentration data is input in step S410 to determine whether there is a potential infection.

At step 414 the procedure determines and stores/outputs infection data. As previously, this may be a simple binary bit indicating the presence or absence of potential infection. However more information may be provided, for example information indicating a degree of infection, a probability of infection, a type of potential infection, infection characterising data, raw and/or processed sensor data, and so forth. In particular, the device may include an optical sensor to provide colourimetric data (S416) and/or a temperature sensor to provide temperature data (S418).

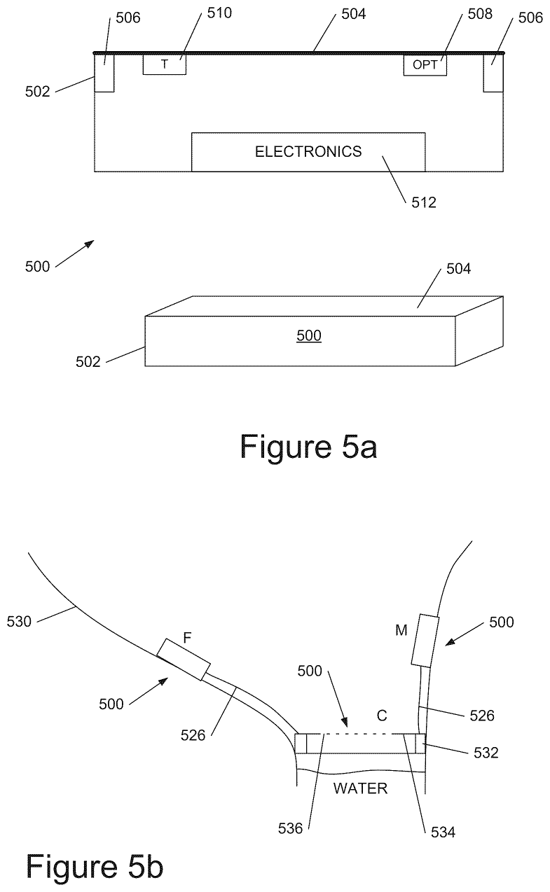

Referring now to FIG. 5a, this shows a preferred embodiment of a sensing device 500, comprising an enclosure 502 with a pressure-sensitive front plate or membrane 504, similar to that previously described with reference to FIG. 1c. The plate 504 is mounted on a set of pressure sensors 506, for example one at each corner of the enclosure. The device includes electronics 512, including communications and a power supply. The device may include an optical (urine colour/optical density) sensor 506 (in which case the plate 504 may be clear), and a temperature sensor 510. A perspective view of the device is also shown.

The device may further include a chemical sensor and/or a gas sensor (not shown). The chemical/gas sensors may be at least partially exposed through the casing. FIG. 5b shows how one or more sensor devices 500 may be positioned within a toilet bowl 530. Position M is suited to male use; position F to female use; central position C is suitable for both. Sensors at M and F may be glued onto the toilet bowl.

The sensor device shown in FIG. 5a is rectangular but for use in position C the sensor device may have a ring-shaped enclosure 532 mounting a plate 534 spanning the ring and having apertures to allow urine to pass through. Where multiple sensor devices are present the sensor at position C may be suspended by linkages 526. One or more of these may be magnetic, and hence detachable; a linkage may also provide an electrical connection. Where multiple sensor devices are present they may be in wired or wireless communication with one another.

The sensor device for use in position C may be mounted on a pivoting mechanism and may include a motor to move the enclosure 532 up, away from the water. A cleaning system may be located above the sensor device. The cleaning system may comprise a reservoir and may be configured to release fluid (e.g. cleaning fluid) to clean the sensor device (e.g. flush away waste). This is because the conventional flush mechanism in a toilet may not be sufficient to remove contaminants from the sensors. The pivoting mechanism may allow cleaning fluid to be collected over the sensors to improve the cleaning action. Once the sensing device has been cleaned, the pivoting mechanism may return the sensing device to its previous position (position C).

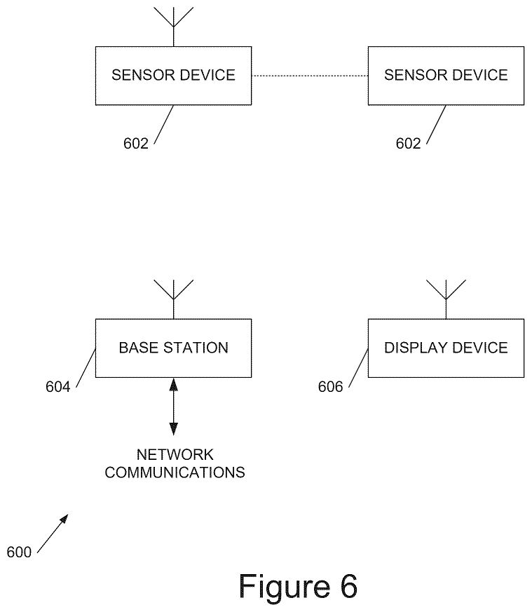

FIG. 6 illustrates a block diagram of an example urine-flow based diagnostic system 600. The system comprises one or more sensor devices 602, coupled to a base station 604 and/or to a local display device 606 to provide alerts and/or sensed data, and the like. The system may be coupled to the cloud via a network.

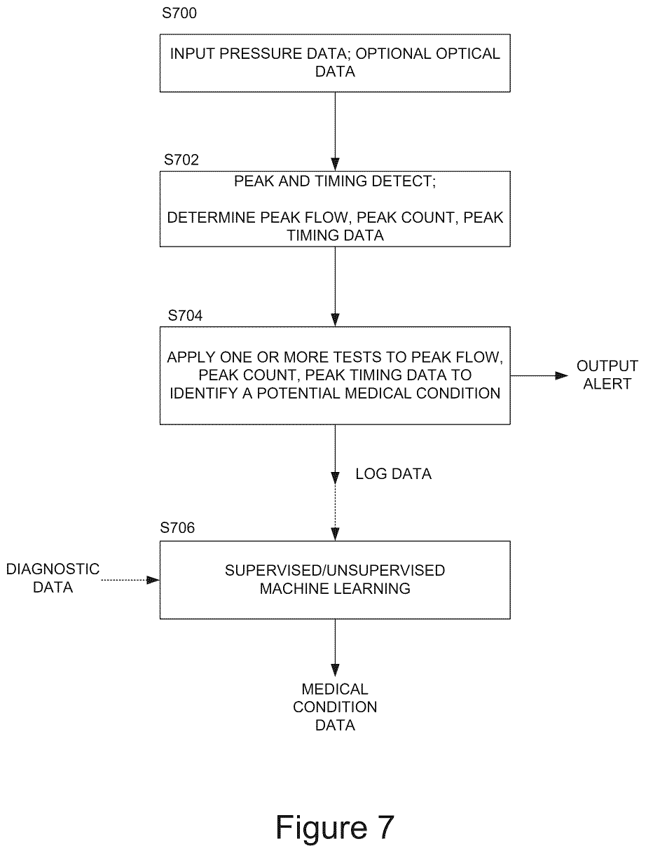

FIG. 7 shows a flow diagram illustrating operation of a urine-flow based diagnostic system according to a preferred embodiment of the invention.

At S700 the procedure captures pressure data as a proxy for urine flow rate, and optionally data for an optical measurement of urine colour/opacity. In embodiments the pressure data may be converted to a flow rate in ml/sec based upon a calibration (which may be linear or non-linear). Although shown as a single step data are captured at intervals of a few milliseconds; the system may be woken up automatically to collect data.

The procedure performs peak and peak-timing detect S702, in one implementation by first smoothing the input data (e.g. filtering in the time domain), and then storing the captured data in a time-series array. A peak may be detected by matching values to either side of a detected maximum; this can be used to determine the duration of a peak. An inter-peak duration may be determined by matching values to either side of a detected minimum. For example in one embodiment the flow rate values in ml/sec are quantised to integer values and then values to either side of a minimum identified (e.g. if the minimum is 2 ml/sec an inter-peak duration may be determined from the interval between flow rates of 3 ml/sec). Once peaks have been identified they may be counted and an inter-peak timing determined, as well as a maximum flow rate for the peak. In embodiments flow rates below a threshold (e.g. 0.2 ml/sec) are disregarded; in embodiments the final two peaks identified are also discarded.

The procedure then processes that data to determine signals of potential infection and of a potential prostate condition (S704). Test as previously described in the Summary of the Invention may be employed. In embodiments multiple signals are required to be present in combination for a positive detection of a potential medical condition. Test applied may include one or more of: Detection of multiple peaks; detection of a low flow rate (<10 ml/sec or <5 ml.sec); detection of an extended time between peaks (>0.5 sec) for infection; detection of less than a threshold or zero time between peaks for a prostate condition; detection of greater than a threshold peak duration (e.g. greater than 10 sec, 15 sec or 20 sec) for a prostate condition.

A result of this processing may be stored and/or output as data indicating the presence of a potential medical condition and, optionally, whether the condition is an infection or a prostate-related condition. Optionally a potential degree of severity of the condition may also be indicated. Additionally or alternatively the raw data from S700 and/or processed data from S702 may be provided. In embodiments an alert and/or the raw/processed data may also be provided on display device 606 and/or communicated to a remote server for further storage/processing.

In embodiments data from one or more of steps S700, S702 is provided to a machine learning procedure S706, for example running in base station 604 and/or on a remote server. The procedure may implement a supervised or unsupervised learning algorithm, for example using supervised learning in combination with data inputted from diagnoses previously made using data from the system, or using unsupervised learning to classify the input data. The machine learning procedure may thus learn to provide improved data indicating the presence of a potential medical condition, and/or differentiation of one or more conditions (such as infection and prostate-related conditions).

Further parameters may be utilised to detect infections or medical conditions. These parameters may include chemical concentration (either in urine or in gas emanating from the urine) and time or frequency of urination.

Whilst the above embodiments discuss the detection of an infection, embodiments may be configured to detect other medical conditions, such as cancer, kidney stones or diabetes. For instance, diabetes may be detected through increased frequency of urination at night and/or increased glucose or ketone concentrations in urine. Equally, cancer, such as prostate cancer, may be detected through an increased concentration of chemicals associated with genes associated with the cancer (e.g. PSA and expressions of TMPRSS2:ERG or PCA3).

As previously described, the device itself may provide a signal or may communicate by means of a wired or wireless connection with a base unit or base station, for example a mobile phone, desktop computer or the like. The skilled person will appreciate that, in the various different aspects of the invention, the infection data may be provided via a network such as a mobile phone network or the internet, for example to alert a carer. Additionally or alternatively, an alarm may be triggered should an infection level greater than the threshold and/or a persistent infection be detected.

The urine-flow based diagnostic system may be configured to connect to one or more further sensing devices. For instance, the urine sensing device may receive measurements taken by the infection sensing systems described with reference to FIGS. 1-3, and vice versa. One device may be configured to trigger measurements in the other. For instance, the infection sensing system may take measurements (e.g. heart rate, skin temperature, skin moisture, etc.) in response to a signal from the urine-flow based diagnostic system that the flow has started (indicating that the user is urinating). Measurements from both devices may therefore be combined for improved identification of medical conditions. The measurements may be processed in one of the devices or may be exported to an external system (e.g. a computer) for processing. Accordingly, whilst the above embodiments are described as diagnostic systems, embodiments may not perform any analysis, and may instead report measurements to an external system for analysis.

The urine-flow based diagnostic system may be configured to reset measurements after use. This reset ensures that measurements from a previous user are not erroneously associated with a new user. The reset may be in response to the system being flushed (either by an external flushing mechanism or via the cleaning mechanism described above). Alternatively, the reset may be via using a magnetic wave from a magnetic tag, an RFID signal from an RFID tag, from a wireless signal (e.g. a Bluetooth signal or a command from a wireless base station), or from a reset input such as a button or pull-chord.

The systems described herein may associate measurements with users through the use of a unique user identifier. This may be input via a command from an external system (e.g. via a wireless interface), or may be input from an RFID tag or magnetic tag that the system is configured to read. This allows the system to be used with different users, and for measurements to be taken over time and associated with the respective user. This may be particularly advantageous for the urine-flow based diagnostic system (although it may be implemented by the infection system of FIGS. 1-3) as multiple users may use the system when it is fitted to a toilet or urinal.

The processing has been described as taking place at the sensing device but the skilled person will appreciate that in embodiments the processing may be partly or substantially wholly at a mobile or fixed computing device with which the sensing device communicates. Thus the device may transmit sensor data, for example via a Bluetooth or other link, to a mobile device implementing a procedure as described above. Alternatively the processing may be distributed, partly on the sensing device and partly on, say, a mobile device.

Further aspects of the invention are defined in the following clauses:

1. An infection sensing system, the system comprising:
a sensor device configured to be fastened onto the skin, the sensor device comprising a plurality of sensors; and
a processor coupled to the sensors;
wherein the sensors comprise at least:
   a temperature sensor to read skin temperature as a proxy for body temperature; and
   a heart rate sensor; and
wherein the processor is configured to:
input data from said sensors and determine data representing body temperature and heart rate; and
identify a combination of:
(i) a greater than threshold temperature fluctuation in said body temperature, and
(ii) a greater than threshold heart rate, and
wherein (i) and (ii) are present for greater than a threshold time duration; and
responsive to said identification, store and/or output data indicating infection.

2. An infection sensing system as defined in clause 1 wherein said sensors further comprise a skin moisture sensor to sense a level of moisture on the surface of the skin, and wherein said processor is configured to identify that a combination of (i) and (ii) and (iii) a greater than threshold skin moisture level, are present for a duration greater than said threshold time duration.

3. An infection sensing system as defined in clause 1 or 2 wherein said sensors further comprise one or more of a chemical sensor or a gas sensor, and wherein said processor is configured to identify that a combination of (i), (ii), optionally (iii), and (iv) a greater than threshold concentration of chemical indicative of a medical condition, are present for a duration greater than said threshold time duration.

4. An infection sensing system as defined in clause 1, 2 or 3 wherein said sensors further comprise an accelerometer, and wherein said processor is further configured to identify, in combination with (i), (ii), optionally (iii), and optionally (iv), a rest state of said body.

5. An infection sensing system as defined in any preceding clause wherein said data indicating infection indicates a degree of infection of said body.

6. An infection sensing system as defined in any preceding clause wherein said sensor device comprises an enclosure with a sensing surface comprising a reduced thickness face or membrane to touch the skin, the device comprising two temperature sensors, a first temperature sensor against said sensing surface to measure the body temperature and second temperature sensor on an external face of said enclosure opposite said sensing surface to measure an environment temperature; wherein said enclosure includes sensing system electronic circuitry mounted on said external face and thermally insulated from said sensing surface; and wherein said processor is configured to compensate a sensed body temperature for said environment temperature.

7. An infection sensing system as defined in any preceding clause wherein said threshold time duration is at least one hour.

8. An infection sensing system as defined in any preceding clause wherein said processor is configured to determine infection responsive to detection of repeated instances of said identification of said combination.

9. An infection sensing system as defined in any preceding clause wherein said processor is configured to:
identify a first combination (i) and (ii) with a first threshold heart rate and a first threshold time duration and a second combination (i) and (ii) with a second threshold heart rate and a second threshold time duration; wherein said second threshold heart rate higher than said first threshold heart rate and wherein said second threshold time duration is shorter than said first threshold time duration; and
categorise an infection into one of at least first and second categories responsive to respective identification of said first and second combinations;
wherein said data indicating infection indicates said category of infection.

10. An infection sensing system as defined in any preceding clause wherein said sensor device comprises an enclosure with a sensing surface comprising a reduced thickness face or membrane to touch the skin and an external face opposite said sensing surface, wherein said enclosure includes sensing system electronic circuitry; and wherein the sensor device further comprises an electrical power supply comprising a thermoelectric power generating device thermally coupled between said sensing surface and said external face to generate electrical power for said electronic circuitry from a temperature difference between the skin and the environment.

11. An infection sensing system as defined in any preceding clause wherein said sensor device comprises a flexible circuit board with an adhesive layer or region to attach the circuit board to the skin, in particular wherein the sensor device is in the form of a plaster.

12. A method of sensing infection using an infection sensing system, in particular as defined in any preceding clause, the method comprising:
measuring body temperature and/or heart rate and skin moisture level; and identifying a combination of:
(i) a greater than threshold temperature fluctuation and/or a combination of:
(ii) a greater than threshold heart rate, and
(iii) optionally a greater than threshold skin moisture level;
wherein (i), (ii), and optionally (iii), are present for greater than a threshold time duration; and
responsive to said identification storing and/or outputting data indicating infection.

13. A non-transitory data carrier carrying processor control code to implement the method of clause 12.

14. A urine-flow based diagnostic system, the system comprising:
a sensor device for use in a toilet bowl or urinal so as to intersect a stream of urine, the sensor device comprising a urine stream flow sensor; and
a processor coupled to said urine stream flow sensor; and
wherein the processor is configured to:
determine, from said urine stream flow sensor, a flow rate parameter dependent on a sensed urine flow; and
responsive to said flow rate parameter, store and/or output data indicating presence of a potential medical condition.

15. A system as defined in clause 14 wherein said processor is further configured to identify, from said flow rate parameter, when a flow of said stream of urine is intermittent to identify presence of said potential medical condition.

16. A system as defined in clause 14 or 15 wherein said processor is configured to identify the presence of a plurality of peaks in said flow rate to identify presence of said potential medical condition.

17. A system as defined in clause 16 wherein said processor is configured to distinguish between the presence of a prostate condition and an infection dependent upon one or both of a number of said detected peaks and a flow rate of said stream of urine.

18. A system as defined in clause 16 or 17 wherein said processor is configured to characterise a timing of said peaks to identify presence of potential infection.

19. A system as defined in clause 18 wherein characterisation of said timing comprises one or more of:
determining a time duration, T1, between one or more of said peaks;
determining a time duration, T2, of a period of reduced or substantially zero flow between one or more of said peaks;
determining a time duration, T3, of one or more of said peaks;
determining a ratio between T2 and T3.

20. A system as defined in clause 18 wherein said characterisation comprises identifying presence of potential infection by identifying when T2 is increased or above a threshold value and/or T3 is reduced or below a threshold value.

21. A system as defined in any one of clauses 14 to 20 wherein said processor is configured to identify when a peak flow rate is less than a threshold to identify presence of said potential medical condition, in particular identify the potential presence of a prostate condition dependent upon a flow rate range of said stream of urine.

22. A system as defined in any one of clauses 14 to 21 wherein said sensor device further comprises a urine stream colour sensor; and wherein said processor is further configured to identify presence of the potential medical condition responsive to a detected degree of colouration of said urine.

23. A system as defined in any one of clauses 14 to 22 wherein:
said sensor device further comprises a chemical sensor, and said processor is further configured to identify presence of the potential medical condition responsive to a detected concentration of a chemical indicative of a medical condition within said urine; and/or
said sensor device further comprises a gas sensor and said processor is further configured to identify presence of the potential medical condition responsive to a detected concentration of a chemical indicative of a medical condition in gas deriving from said urine.

24. A system as defined in any one of clauses 14 to 23 further comprising an alert device to alert the urinating person to the presence of the potential medical condition. 25. A system as defined in any one of clauses 14 to 24 wherein said urine stream flow sensor comprises a plurality of spatially distributed flow sensors, and wherein said processor is configured to combine data from said distributed flow sensors to sense said urine flow.

26. A system as defined in clause 25 wherein said processor is configured to distinguish between genders for said urine flow dependent upon a sensed spatial distribution of said urine flow.

27. A system as defined in clause 25 or 26 wherein said sensor device comprises an enclosure with a sensing surface comprising a moveable plate or membrane mounted on a set of pressure sensors, wherein said set of pressure sensors comprises said distributed flow sensors.

28. A system as defined in any one of clauses 14 to 27 wherein said sensor device comprises a ring-shaped enclosure mounting a pressure-sensing surface comprising a plate or membrane having a set of apertures.

29. A system as defined in any one of clauses 14 to 28 further comprising a cleaning system comprising a reservoir configured to hold fluid and a release mechanism for releasing at least some of the fluid to clean at least part of the sensor device.

30. A system as defined in clause 29 wherein the processor is configured to control the release mechanism to release the fluid after urine has been detected.

31. A system as defined in clause 29 or 30 further comprising a fluid collection mechanism configured to collect fluid over one or more sensing apparatus of the system after the fluid has been released from the reservoir.

32. A system as defined in any one of clauses 14 to 31 wherein the processor is configured to record a time at which the urine flow is detected and, responsive to one or more of an increased frequency of urination or an increased frequency of urination during a predetermined period of the day-night cycle, store and/or output data indicating the presence of a medical condition.

33. A system as defined in any one of clauses 14 to 32 wherein the system comprises an input means and wherein the processor is configured to identify the presence of a new user in response to an input from a user and, in response to the identification of the new user, establish a new set of measurements for the new user.

34. A system as defined in clause 33 wherein the processor is configured to identify the new user in response to receipt of a unique user identifier and to associate any subsequent measurements and identifications of medical conditions with the unique user identifier until the presence of another user is identified.

35. An infection sensing system according to any one of clauses 1-13 further comprising a wireless communication module, wherein the processor is configured to trigger one or more measurements by one or more of the sensors in response to an input from a urine-flow based diagnostic system according to any one of clauses 14-34 indicating that the user of the infection sensing system is urinating.

36. A method of sensing presence of a potential medical condition using a urine sensing device including a urine stream flow sensor, the method comprising:
determining, from said flow sensor, a flow rate parameter dependent on sensed urine flow rate; and
responsive to said flow rate parameter, storing and/or outputting data indicating presence of a potential medical condition.

37. A method as defined in clause 36 further comprising identifying from said flow rate parameter the presence of a plurality of peaks in said urine flow rate, and determining presence of potential infection responsive to said identifying.

38. A non-transitory data carrier carrying processor control code to implement the method of clause 36 or 37.

No doubt many other effective alternatives will occur to the skilled person. It will be understood that the invention is not limited to the described embodiments and encompasses modifications apparent to those skilled in the art lying within the spirit and scope of the claims appended hereto.

1. An infection sensing system, the system comprising:
a sensor device configured to be fastened onto skin, the sensor device comprising:
a plurality of sensors;
an enclosure with a sensing surface, the sensing surface having a reduced thickness face or membrane to touch the skin, and an external face opposite said sensing surface, wherein the enclosure includes sensing system electronic circuitry mounted on the enclosure and thermally insulated from the sensing surface;
an electrical power supply comprising a thermoelectric power generating device thermally coupled between the reduced thickness face or membrane and the external face of the enclosure to generate electrical power for the electronic circuitry from a temperature difference between the skin and the environment; and
a processor coupled to the sensors;
wherein the sensors comprise at least:
a first temperature sensor thermally coupled to the reduced thickness face or membrane to read skin temperature as a proxy for body temperature;
a second temperature sensor on the external face of the enclosure to measure an environment temperature;
a heart rate sensor configured to detect pressure on the reduced thickness face or membrane to detect a heartbeat; and
a skin moisture sensor provided on the reduced thickness face or membrane to sense a level of moisture on the surface of the skin; and
a gas sensor configured to measure one or more chemicals indicative of a medical condition in the air adjacent to the skin, wherein the gas sensor is provided on the external face of the enclosure; and
a reflective SpO2 sensor to perform a one-sided measurement of oxygen saturation; and
wherein the processor is configured to:
input time series data from said sensors and determine data representing body temperature, heart rate, skin moisture level and SpO2 level;
compensate the data representing body temperature for the environment temperature; and
input the data representing body temperature, heart rate, skin moisture level and SpO2 level into a multivariate time-series analysis neural network trained to perform temporal pattern recognition to identify a combination of:
(i) a greater than threshold temperature fluctuation in said body temperature,
(ii) a greater than threshold heart rate,
(iii) a greater than threshold skin moisture level,
(iv) a greater than threshold concentration of chemical indicative of a medical condition in the air adjacent to the skin, and
(v) a reduction in SpO2 level,
wherein (i), (ii), (iii), (iv) and (v) are present for greater than a threshold time duration; and
v) a variation in measured SpO2 level; and
responsive to said identification, store and/or output data indicating infection;
wherein the multivariate time-series analysis neural network is configured to process time series data that extends over a duration of at least the threshold time duration, wherein the threshold duration is at least one hour; and
wherein the time series data representing body temperature, heart rate, skin moisture level and SpO2 level input into the multivariate time-series analysis neural network comprises data that comprises simultaneous measurements for at least the heart rate and SpO2 level, wherein the simultaneous measurements are simultaneous to better than 100 milliseconds.

2. The infection sensing system as claimed in claim 1, wherein the simultaneous measurements are simultaneous to better than 10 milliseconds.

3. The infection sensing system of claim 1, wherein the processor comprises a master-slave processor system, comprising a master processor and one or more slave processors, wherein the one or more slave processors are coupled to the sensors, wherein the master processor is configured to implement the multivariate time-series analysis neural network, and wherein the master processor is configured to control the one or more slave processors to perform the simultaneous measurements.

4. The infection sensing system of claim 3, wherein the master processor is implemented on a smartphone or laptop computer, and wherein the one or more slave processors are implemented on a wearable device comprising the plurality of sensors and the enclosure.

5. The infection sensing system of claim 1, wherein the multivariate time-series analysis neural network comprises a temporal convolutional network or recurrent neural network.

6. The infection sensing system of claim 1, wherein said processor is further configured to:
identify a first combination (i) and (ii) with a first threshold heart rate and a first threshold time duration and a second combination (i) and (ii) with a second threshold heart rate and a second threshold time duration; wherein said second threshold heart rate is higher than said first threshold heart rate and wherein said second threshold time duration is shorter than said first threshold time duration; and
categorize an infection into one of at least first and second categories responsive to respective identification of said first and second combinations;
wherein said data indicating infection indicates said category of infection.

7. The infection sensing system of claim 1, wherein said processor is further configured to:
determine an integrated temperature variation over time; and
provide the integrated temperature variation to the multivariate time-series analysis neural network to represent the temperature fluctuation.

8. The infection sensing system of claim 1, wherein the gas sensor comprises a compound semiconductor gas sensor configured to measure one or more of: NO, NO2 and CO2.

9. A method of sensing infection using the infection sensing system of claim 1, the method comprising:
measuring body temperature, heart rate and skin moisture level; and
inputting the body temperature, heart rate and skin moisture level into a neural network trained to identify a combination of:
(i) a greater than threshold temperature fluctuation,
(ii) a greater than threshold heart rate,
(iii) a greater than threshold skin moisture level;
(iv) a greater than threshold concentration of chemical indicative of a medical condition in the air adjacent to the skin,
wherein (i), (ii), and (iii), are present for greater than a threshold time duration;
responsive to said identification storing and/or outputting data indicating infection.

10. A non-transitory data carrier carrying processor control code to implement the method of claim 9.

11. An infection sensing system, the system comprising:
a wearable sensor device configured to be fastened onto skin, the wearable sensor device comprising:
a plurality of sensors; and
a processor coupled to the sensors;
wherein the sensors comprise at least:
a temperature sensor to read skin temperature as a proxy for body temperature;
a heart rate sensor; and
a reflective SpO2 sensor to perform a one-sided measurement of oxygen saturation; and
wherein the processor is configured to:
input time series data from said sensors and determine data representing body temperature, heart rate, and SpO2 level; and
process the data representing body temperature, heart rate, and SpO2 level using a multivariate time-series analysis neural network trained to perform temporal pattern recognition, to generate an output indicating infection; and
wherein the multivariate time-series analysis neural network is configured to process time series data that extends over a duration of at least the threshold time duration, wherein the threshold duration is at least one hour; and
wherein the time series data representing body temperature, heart rate, and SpO2 level input into the multivariate time-series analysis neural network comprises data that comprises simultaneous measurements for at least the heart rate and SpO2 level, wherein the simultaneous measurements are simultaneous to better than 100 milliseconds.

12. The infection sensing system of claim 11, wherein the simultaneous measurements are simultaneous to better than 10 milliseconds.

13. The infection sensing system of claim 11, wherein:
the processor comprises a master-slave processor system, comprising a master processor and one or more slave processors;
wherein the one or more slave processors are implemented on a wearable device comprising the plurality of sensors and are coupled to the sensors;
wherein the master processor is implemented on a smartphone or laptop computer and is configured to implement the multivariate time-series analysis neural network; and
wherein the master processor is configured to control the one or more slave processors to perform the simultaneous measurements.

14. The infection sensing system of claim 11, wherein the sensors of the wearable device further comprise:
a compound semiconductor gas sensor configured to measure a level of NO emitted by a wearer of the wearable device; or
a compound semiconductor gas sensor configured to measure a level of NO2 emitted by a wearer of the wearable device; and
wherein the input time series data further comprises the level of NO or the level of NO2; and
wherein the multivariate time-series analysis neural network is further configured to process the level of NO or the level of NO2 to generate the output indicating infection.

15. The infection sensing system of claim 11, wherein the sensors of the wearable device further comprise a compound semiconductor gas sensor configured to measure a level of CO2 emitted by a wearer of the wearable device;
wherein the input time series data further comprises the level of CO2; and
wherein the multivariate time-series analysis neural network is further configured to process the level of CO2; to generate the output indicating infection.

16. The infection sensing system of claim 11, wherein the sensors of the wearable device further comprise a skin moisture sensor configured to measure a level of sweat of a wearer of the wearable device;
wherein the input time series data further comprises the level of sweat; and
wherein the multivariate time-series analysis neural network is further configured to process the level of sweat to generate the output indicating infection.

17. The infection sensing system of claim 11, wherein the multivariate time-series analysis neural network comprises a temporal convolutional network or recurrent neural network.

18. A method of infection sensing using a system comprising:
a wearable sensor device configured to be fastened onto skin, the sensor device comprising:
a plurality of sensors; and
a processor coupled to the sensors;
wherein the sensors comprise at least:
a first temperature sensor to read skin temperature as a proxy for body temperature;
a heart rate sensor configured to detect pressure on the reduced thickness face or membrane to detect a heartbeat; and
a reflective SpO2 sensor to perform a one-sided measurement of oxygen saturation;
the method comprising:
inputting time series data from said sensors and determine data representing body temperature, heart rate, and SpO2 level; and
processing the data representing body temperature, heart rate, and SpO2 level using a multivariate time-series analysis neural network trained to perform temporal pattern recognition, to generate an output indicating infection; and
wherein the multivariate time-series analysis neural network processes time series data that extends over a duration of at least the threshold time duration, wherein the threshold duration is at least one hour; and
wherein the method further comprises measuring making simultaneous measurements of at least the heart rate and SpO2 level, wherein the simultaneous measurements are simultaneous to better than 100 milliseconds.
